(19) United States
(12) Patent Application Publication
(10) Pub. No.: US 2019/0351195 A1
(43) Pub. Date: Nov. 21, 2019

(54) MEDICAL CATHETER AND METHOD FOR MANUFACTURING MEDICAL TUBE

(71) Applicant: OLYMPUS CORPORATION, Tokyo (JP)

(72) Inventors: Kohei OGUNI, Tokyo (JP); Ichiro HORIUCHI, Ina-shi (JP)

(73) Assignee: OLYMPUS CORPORATION, Tokyo (JP)

(21) Appl. No.: 16/527,255

(22) Filed: Jul. 31, 2019

Related U.S. Application Data
(63) Continuation of application No. PCT/JP2018/004686, filed on Feb. 9, 2018.

(30) Foreign Application Priority Data
Feb. 15, 2017 (JP) 2017-025983

(51) Int. Cl. A61M 25/09; A61M 25/00

(52) U.S. Cl. CPC A61M 25/09; A61F 2/95; A61M 25/0015; A61M 25/0052

(57) ABSTRACT

A medical catheter includes: a sheath having a guide wire lumen through which a guide wire is passed and a guide wire port formed in an outer peripheral surface, the guide wire port communicating with the guide wire lumen; a sloped portion that is a portion of the sheath dropped into the guidewire lumen, the sloped portion being provided continuously from an inner wall of the guide wire lumen through the guide wire port to the outer peripheral surface; and a reinforcing portion that is a portion of the sheath softened or melted to be deformed by heating, the reinforcing portion being provided to a proximal end side of the sloped portion in the guide wire lumen, wherein a height of the reinforcing portion is 30% or more of an inner diameter of the guide wire lumen.

### CROSS REFERENCE TO RELATED APPLICATIONS

This application is a continuation application based on a PCT Patent Application No. PCT/JP2018/004686, filed on Feb. 9, 2018, whose priority is claimed on Japanese Patent Application No. 2017-025983, filed on Feb. 15, 2017. The contents of both the PCT Application and the Japanese Application are incorporated herein by reference.

### BACKGROUND

### Technical Field

The present invention relates to a medical catheter, and more particularly to a medical catheter through which a guide wire is passed and used, and a method of manufacturing a medical tube used for the medical catheter.

### Background Art

Conventionally, a medical catheter (hereinafter, may be simply referred to as a “catheter”) having a guide wire lumen through which a guide wire is passed is known. In a general catheter placement procedure, first, the distal end of the guide wire is placed at the intended site in the patient's body via the forceps channel of an endoscope. The proximal end of the guidewire protruding from the proximal end of the forceps channel is then pushed into the guidewire lumen of the catheter (backloading) and the catheter is advanced along the guidewire. This allows the distal end of the catheter to be placed at an intended site.

In order to insert the catheter in a condition that the distal end of the guide wire is fixed at the intended site, the length of a guide wire part protruding from the proximal end of the forceps channel needs to be longer than the entire length of the catheter. If the length of the protruding part of the guidewire is shorter than the distance between the distal end opening and the proximal end opening of the guidewire lumen, it is difficult for the operator to keep the guidewire in the intended position during catheter insertion. Therefore, it also becomes difficult to position the distal end of the catheter at the intended position.

In the case of using a catheter in which the openings of the guide wire lumen are provided at the distal end portion and the proximal end portion of the catheter, the length of the guide wire protruding from the forceps channel becomes longer than the entire length of the catheter, and the operation becomes complicated. On the other hand, it has been proposed and has been put to practical use that a proximal end opening of the guide wire lumen (hereinafter sometimes referred to as a “guide wire port”) be provided on the outer peripheral surface in the middle of the whole length of the catheter.

In a catheter provided with a guide wire port on the outer peripheral surface of the longitudinal middle portion of the catheter, the axis of the proximal end opening provided on the outer peripheral surface and that of the guide wire lumen make an angle with each other such that the extending directions of both axes are different. Therefore, the guide wire may not smoothly come out of the proximal end opening of the guide wire lumen.

In regard to this problem, in Published Japanese Translation No. 2008-509726 of the PCT International Publication, it is described that part of the outer peripheral surface of a catheter is cut out into a flap shape and the flap is pushed into the guide wire lumen. This depressing of the flap makes a proximal end opening of the guide wire lumen and the depressed flap becomes a slope connecting the guide wire lumen and the proximal end opening. By placing the backloaded guide wire along the slope, the guide wire can be smoothly protruded from the proximal end opening.

Although the above-described structure has a certain effect, when this structure is applied with the actual dimensions of the catheter, the guide wire may not necessarily move along the slop.

### SUMMARY

A medical catheter includes: a sheath having a guide wire lumen through which a guide wire is passed and a guide wire port formed in an outer peripheral surface, the guide wire port communicating with the guide wire lumen; a sloped portion that is a portion of the sheath depressed into the guidewire lumen, the sloped portion being provided continuously from an inner wall of the guide wire lumen through the guide wire port to the outer peripheral surface; and a reinforcing portion that is a portion of the sheath softened or melted to be deformed by heating, the reinforcing portion being provided to a proximal end side of the sloped portion in the guide wire lumen, wherein a height of the reinforcing portion is 30% or more of an inner diameter of the guide wire lumen.

The reinforcing portion may be formed of the same material as the sheath.

A rising position of the sloped portion may be positioned on the distal end side with respect to the distal end of the guide wire port.

A method of manufacturing a medical tube, includes: a process of preparing a material tube having a lumen; a first step of cutting an outer peripheral surface of the material tube and depressing the cut part of the outer peripheral surface into the lumen to form a guide wire port communicating with the lumen; a second step of inserting a core into the lumen and causing it to protrude from the guide wire port; a third step, in a state in which the core is positioned in the lumen after the second step, of heating the material tube and connecting the part of the outer peripheral surface depressed into the lumen with an inner wall of the lumen.

According to the medical catheter and the medical tube manufacturing method of the present invention, a backloaded guidewire can be more smoothly protruded from the proximal end side opening formed on the outer peripheral surface.

### BRIEF DESCRIPTION OF THE DRAWINGS

FIG. 1 is a perspective view showing a stent mounted on a medical catheter according to a first embodiment of the present invention.

FIG. 2 is a cross-sectional view schematically showing the medical catheter.

FIG. 3 is a partially enlarged view showing a second tube of the medical catheter.

FIG. 4 is a cross-sectional view taken along the line I-I of FIG. 3.

FIG. 5 is a cross-sectional view showing a double lumen tube which is a material of the second tube.

FIG. 6 is a cross-sectional view showing a process of the method of manufacturing the second tube.

FIG. 7 is a cross-sectional view showing another example of one process of manufacturing method of the second tube.

FIG. 8 is a cross-sectional view showing a process of manufacturing the second tube.

FIG. 9 is a cross-sectional view showing a process of manufacturing the second tube.

FIG. 10 is a cross-sectional view showing a process of manufacturing method of the second tube.

FIG. 11 is a cross-sectional view showing a second tube according to a modified example of the present invention.

FIG. 12 is a cross-sectional view showing a second tube according to a modified example of the present invention.

### DETAILED DESCRIPTION OF THE PREFERRED EMBODIMENTS

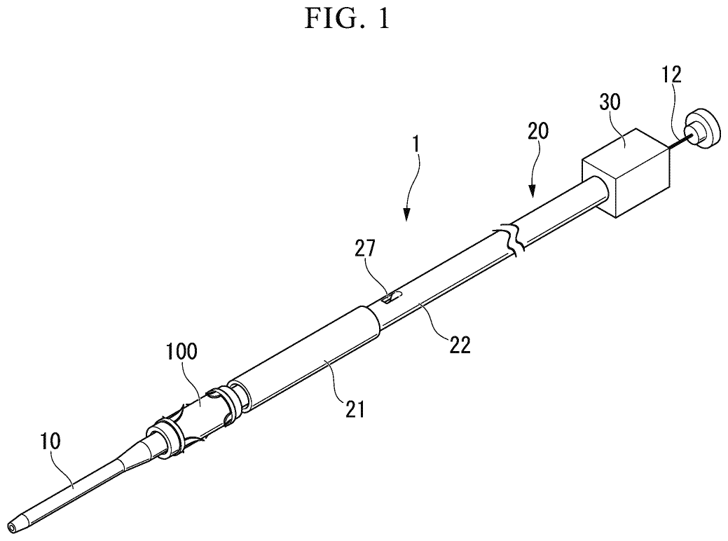

An embodiment of the present invention will be described with reference to FIGS. 1 to 8.

FIG. 1 is a perspective view showing a state in which a stent 100 is attached to a medical catheter 1 according to the present embodiment. The catheter 1 includes an elongated catheter body 10, a tube 20 through which the catheter body 10 is passed, and an operation unit 30 provided at the proximal end of the tube 20.

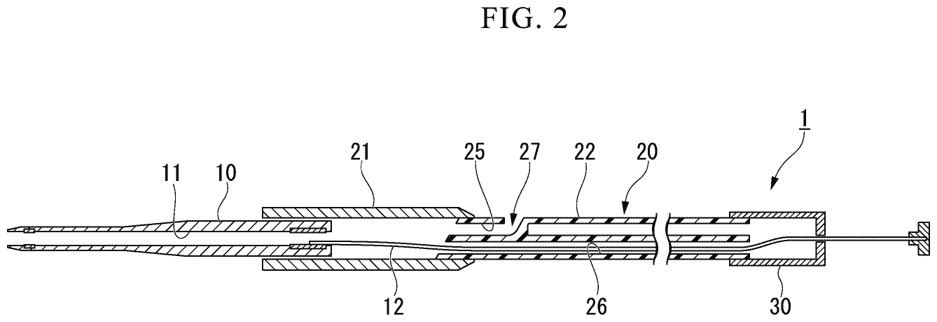

FIG. 2 is a cross-sectional view schematically showing the catheter 1.

The catheter body 10 is a tubular member formed of resin or the like and has a catheter lumen 11 having an inner diameter through which a guide wire can be passed. The catheter lumen 11 extends the entire length of the catheter body 10 and is open at the distal end and the proximal end of the catheter body 10. An operation wire 12 is connected to the proximal end of the catheter body 10.

The tube 20 is a tubular member having an inner diameter larger than the outer diameter of the catheter body 10. The tube 20 is configured by connecting a first tube 21 on the distal end side and a second tube (sheath) 22 on the proximal end side. The first tube 21 has one lumen through which the proximal end of the catheter body 10 can enter. The second tube 22 has two lumens, a first lumen (guide wire lumen) 25 through which a guide wire is passed and a second lumen 26 through which the operation wire 12 is passed. A guide wire port 27 communicating with the first lumen 25 is formed on the outer peripheral surface of the second tube 22.

The operation unit 30 is formed of, for example, a resin or the like. The operation wire 12 connected to the catheter body 10 extends through the second lumen 26 of the tube 20 to the operation unit 30 and protrudes from the proximal end of the operation unit 30. While holding the proximal end of the operation wire 12, the user can advance and retract the tube 20 with respect to the catheter body 10 by holding the operation unit 30 and advancing and retracting the operation wire 12. The inner diameter of the tube 20 is smaller than the outer diameter of the stent 100. Therefore, by advancing the tube 20, the stent 100 mounted on the catheter body 10 can be pushed out and indwelled in a body.

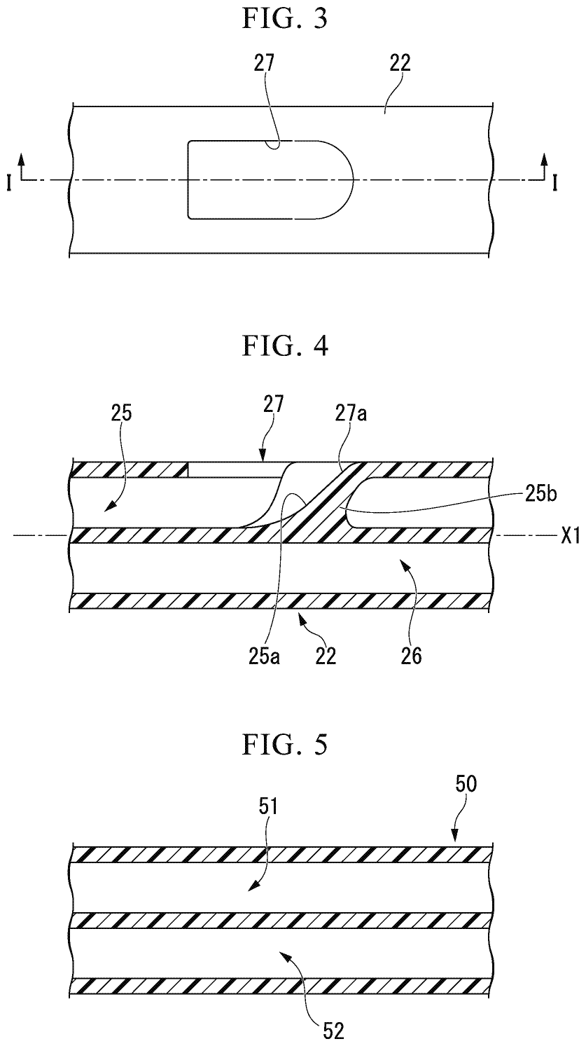

FIG. 3 is a partial enlarged view showing the vicinity of the guide wire port 27 of the second tube 22, and FIG. 4 is a cross-sectional view taken along the line I-I of FIG. 3. The shape of the guide wire port 27 can be set as appropriate.

As shown in FIG. 4, in the first lumen 25 near the guide wire port 27, a sloped portion 25a is provided. The sloped portion 25a reaches the proximal end portion 27a through the guide wire port 27 from the part near the central axis X1 of the second tube 22 in the inner wall of the first lumen 25. The sloped portion 25a is continuous with the outer peripheral surface of the second tube 22. On the proximal end side of the sloped portion 25a, a reinforcing portion 25b that reinforces the sloped portion 25a is provided integrally with the sloped portion 25a, the sloped portion 25a is made from the same material as the second tube 22.

A method of manufacturing the second tube (medical tube) 22 of the present embodiment provided with the guide wire port 27, the sloped portion 25a, and the reinforcing portion 25b will be described.

First, a double lumen tube (material tube) 50 as shown in FIG. 5 is prepared as a material of the second tube. The double lumen tube 50 has a pre-first lumen 51 (lumen) which will be the first lumen 25 after the processing, and a second lumen 26. The pre-first lumen 51 and the second lumen 26 both extend parallel to the axis of the double lumen tube 50.

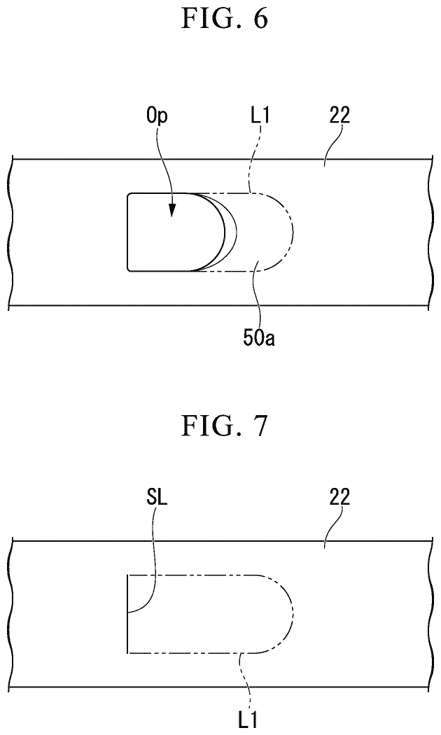

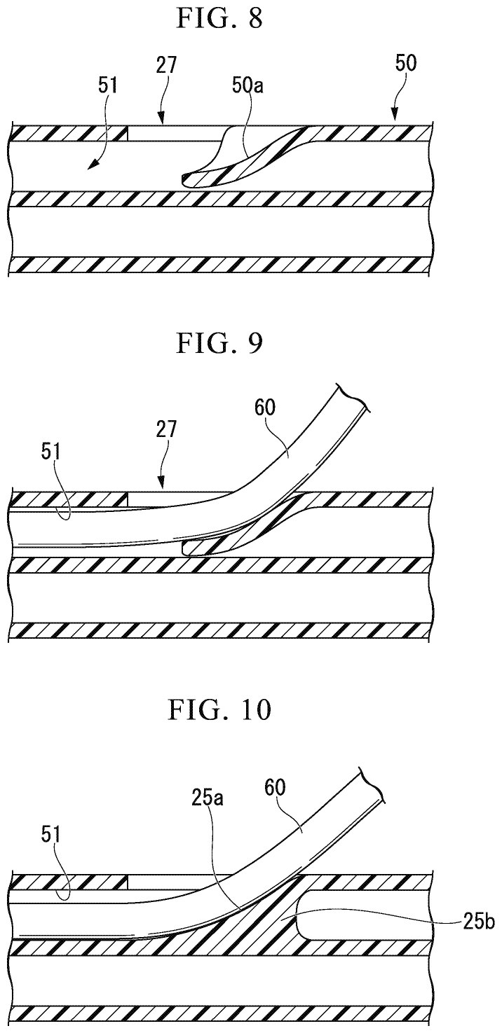

Next, the outer peripheral surface of the double lumen tube 50 is cut at a position where the guide wire port will be formed so that the outer peripheral surface is partially removed, and an opening Op communicating with the pre-first lumen 51 as shown in FIG. 6 is formed. Next, a portion 50a of the outer peripheral surface is depressed, or pushed down, along the contour L1 of the guide wire port 27 to be formed into the pre-first lumen 51 as shown in FIG. 8 (first step). The shape of the incision can be set as appropriate. For example, as shown in FIG. 7, only the linear incision SL may be formed without removing the outer peripheral surface to form the opening Op.

When the first step is completed, a guide wire port 27 communicating with the pre-first lumen 51 is formed on the outer peripheral surface of the double lumen tube 50.

Next, a core 60 is inserted into the pre-first lumen 51 from the side on the distal end of the second tube and is protruded from the guide wire port 27 as shown in FIG. 9 (second step). The diameter of the core 60 is larger than the diameter of the guide wire to be used but not too large in the difference (clearance) with the inner diameter of the pre-first lumen.

In the present embodiment, the material of the core 60 is not limited to metal. For example, a thermosetting resin or the like may be used as the material of the core 60 as long as the shape can be sufficiently maintained at the heating temperature in the third step described later.

Next, with the core passed, the periphery of the guide wire port of the double lumen tube 50 is heated (third step). The heating temperature in the third step may be at least a temperature at which the material of the double lumen tube 50 is softened, or may be temporarily equal to or higher than the melting point of the material of the double lumen tube 50.

In the third step, the core 60 defines the range in which the shape of the portion 50a of the outer peripheral surface and the double lumen tube 50 around it can be deformed. Therefore, in a state where a space through which the guide wire passes in the pre-first lumen 51 is secured, the portion 50a of the outer peripheral surface and the double lumen tube 50 around it are softened or melted to be deformed by heating.

When the third step is completed, as shown in FIG. 10, the sloped portion 25a continuous from the inner wall of the pre-first lumen 51 to the outer peripheral surface of the double lumen tube 50 is formed in the pre-first lumen 51. A reinforcing portion 25b, which is made from the same material as the sloped portion 25a and is integrated with the sloped portion 25a, is formed in the pre-first lumen 51. The sloped portion 25a is thicker than the portion 50a of the outer peripheral surface before melting because the reinforcing portion 25b is combined.

Thereafter, when the core 60 is removed from the pre-first lumen 51, the second tube 22 is completed. The second tube 22 has the first lumen 25 and the second lumen 26, and the first lumen 25 has the guide wire port 27, the sloped portion 25a, and the reinforcing portion 25b.

In the technique described in the above mentioned Published Japanese Translation No. 2008-509726 of the PCT International Publication, a part of the outer peripheral surface is depressed into the lumen to form a slope, and the formed slope is not integrated with the inner wall of the lumen. Furthermore, depressed part of the outer peripheral surface is stretched and thinned as it is pushed down into the lumen, and the strength is also reduced.

Therefore, there were cases in which the proximal end of the backloaded guidewire may peel off the formed slope proximally or pierce it. The proximal end of the guidewire may not project smoothly from the guidewire port.

In the catheter 1 of the present embodiment, a sloped portion 25a continuous from the inner wall of the first lumen 25 to the outer peripheral surface of the double lumen tube 50 and a reinforcing portion 25b that reinforces the sloped portion 25a are provided in the vicinity of the guide wire port 27. The sloped portion 25a integrated with the inner wall is not flipped by the backloaded guide wire and is prevented by the reinforcing portion 25b from being pierced. Therefore, the backloaded guide wire can be suitably protruded from the guide wire port 27 along the sloped portion 25a.

Moreover, according to the manufacturing method of the medical catheter tube of the present embodiment described above, only by inserting the core through the existing double lumen tube and heating it, a medical tube can be manufactured simply and efficiently. The medical tube has a sloped portion and a reinforcing portion and a guide wire port formed on the outer peripheral surface.

As described above, although an embodiment of the present invention has been described, the technical scope of the present invention is not limited to the above-described embodiment. It is possible to change the combination of components and make various changes to or delete each component in the range which does not deviate from the meaning of the present invention.

For example, in the present invention, the shapes of the sloped portion and the reinforcing portion are not limited to those shown in the above embodiment.

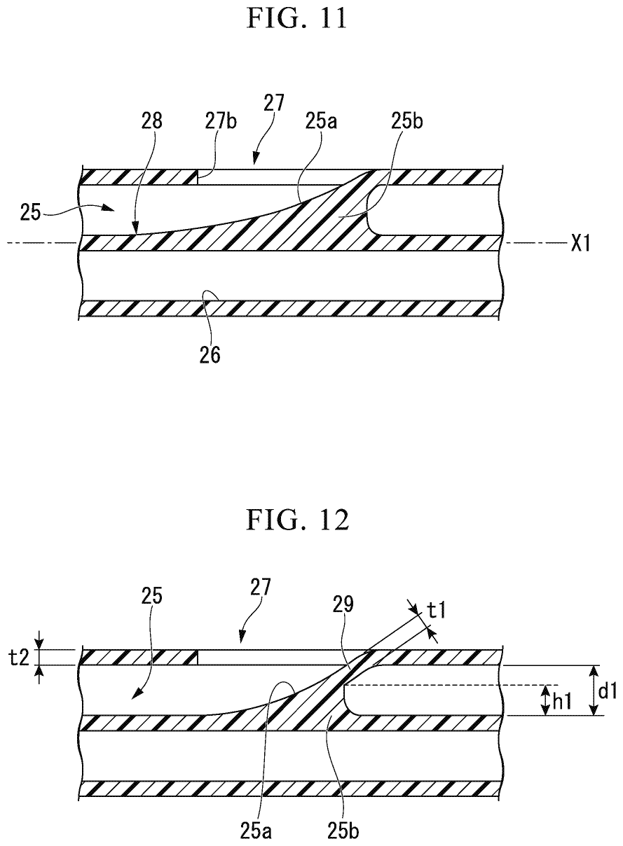

In the modified example shown in FIG. 11, the rising position 28 of the sloped portion 25a is located more distal to the peripheral edge 27b of the most distal end side of the guide wire port 27. Here, the rising position 28 of the sloped portion 25a is a position where the inner wall of the first lumen 25 becomes nonparallel to the central axis X1 of the second tube 22. The partition wall between the first lumen 25 and the second lumen 26 starts to become thicker from the rising position 28. The portion where the guide wire port 27 is formed has a lower rigidity than other portions because the outer peripheral surface is removed, and it is easy to cause buckling or the like. However, by positioning the rising position 28 more distal to the guide wire port 27, the sloped portion 25a and the reinforcing portion 25b can compensate for the decrease in rigidity and suppress the occurrence of buckling or the like.

In the modification shown in FIG. 12, the reinforcing portion 25b is provided only in a partial region in the first lumen 25. When the guide wire port 27 is viewed in plan as in the I-I line shown in FIG. 3, in the cross section of the second tube 22 in a plane including the central axis X1, the height h1 of the reinforcing portion 25b shown in FIG. 12 is preferably 30% or more of the inner diameter d1 of one lumen 25, and more preferably 50% or more.

In a portion where the reinforcing portion 25b is not provided, the thickness of the sloped portion 25a is substantially the same as the thickness of the portion 50a of the outer peripheral surface before melting, and the rigidity is not necessarily sufficient. However, usually, the diameter of the guide wire is about 80% of the inner diameter of the guide wire lumen, and even in the thinnest case, it is about 55% of the inner diameter of the guide wire lumen. If the height h1 of the reinforcing portion 25b is 30% or more of the inner diameter d1 of the first lumen 25, the proximal end of the back-loaded guide wire easily contacts the region of the sloped portion reinforced by the reinforcing portion. As a result, it can be sufficiently suppressed that the guide wire penetrates the sloped portion.

In the present invention, the position of a top part 29 defining the height of the reinforcing portion 25b is defined as a point where the thickness t1 (see FIG. 12) of the sloped portion 25a, which is a dimension measured in the direction perpendicular to the surface direction of the sloped portion 25a, starts to be larger than the thickness t2 of the wall surface on the outer peripheral surface side of the first lumen 25 in the region where the guide wire port is not provided.

Moreover, although the above embodiment has described the example which the second tube 22 includes the second lumen, the second lumen is not indispensable, and it may not be provided if it is not necessary due to the configuration of the medical catheter to be applied.

In this case, the medical tube may be manufactured by the above-described manufacturing method using a material tube having only a pre-first lumen.

Furthermore, the catheter of the present invention is not limited to one in which the second tube is formed using a double lumen tube. For example, only the portion of the guide wire port including the sloped portion and the reinforcement portion may be formed by injection molding using resin and a tube may be connected to before and after the portion of the guide wire port to make the second tube 22 or the tube 20.

In addition, even when the second tube is manufactured using a double lumen tube, the manufacturing method is not limited to that described above. For example, the double lumen tube may be heated after the member serving as the reinforcing portion is disposed in advance in the first lumen at a place more the proximal to the depressed outer peripheral surface in the first lumen. In this case, the material of the reinforcing portion can be different from the material of the sloped portion.

The present invention is widely applicable to medical catheters and medical tube manufacturing methods, and allows a backloaded guidewire to be more smoothly protruded from the proximal end side opening formed on the outer peripheral surface.

What is claimed is:

1. A medical catheter comprising:
a sheath having a guide wire lumen through which a guide wire is passed and a guide wire port formed in an outer peripheral surface, the guide wire port communicating with the guide wire lumen;
a sloped portion that is a portion of the sheath depressed into the guidewire lumen, the sloped portion being provided continuously from an inner wall of the guide wire lumen through the guide wire port to the outer peripheral surface; and
a reinforcing portion that is a portion of the sheath softened or melted to be deformed by heating, the reinforcing portion being provided to a proximal end side of the sloped portion in the guide wire lumen,
wherein a height of the reinforcing portion is 30% or more of an inner diameter of the guide wire lumen.

2. The medical catheter according to claim 1, wherein the reinforcing portion is formed of the same material as the sheath.

3. The medical catheter according to claim 1, wherein a rising position of the sloped portion is positioned on the distal end side with respect to the distal end of the guide wire port.

4. A method of manufacturing a medical tube, comprising:
a process of preparing a material tube having a lumen;
a first process of cutting an outer peripheral surface of the material tube and dropping a part of the outer peripheral surface into the lumen to form a guide wire port communicating with the lumen;
a second step of inserting a core into the lumen and causing it to protrude from the guide wire port;
a third step, in a state in which the core is positioned in the lumen after the second step, of heating the material tube and connecting the part of the outer peripheral surface depressed into the lumen with an inner wall of the lumen.
